(19) United States
(12) Patent Application Publication
(10) Pub. No.: US 2004/0224367 A1
(43) Pub. Date: Nov. 11, 2004

(54) Method of immobilizing cell

(76) Inventors: Nobuhiko Ozaki, Ikoma-shi (JP); Hiroaki Oka, Hirakata-shi (JP)

Correspondence Address: MCDERMOTT WILL & EMERY LLP, 600 13TH STREET, N.W., WASHINGTON, DC, 20005-3096, US

(21) Appl. No.: 10/694,441

(22) Filed: Oct. 28, 2003

(30) Foreign Application Priority Data
Oct. 28, 2002 (JP) 2002-313063

(51) Int. Cl. G01N 33/53; G01N 33/567

(52) U.S. Cl. 435/7.2; 435/325

(57) ABSTRACT

A method of the immobilization is provided in which a cell can be immobilized with high accuracy in a state where its activity is maintained, even in a minute region, or a region that constitutes a complicated pattern. A method of the immobilization of a cell in which a cell is immobilized in a desired region among the surface of a substrate, which includes the step of forming a masking layer in a region except for the aforementioned desired region on the surface of the aforementioned substrate (St2), the step of immobilizing the aforementioned cell through bringing a solution containing the aforementioned cells into contact with the surface of the aforementioned substrate and the surface of the aforementioned masking layer (St4, St5), and the step of adjusting the pH of the aforementioned solution to give the condition which permits separation of the aforementioned masking layer from the aforementioned substrate without loss of the activity of the aforementioned cell (St6).

### BACKGROUND OF THE INVENTION

1. Field of the Invention

The present invention relates to a method of the immobilization of a cell in which a cell is immobilized in a desired region on the surface of a substrate.

2. Description of the Related Art

In recent years, elucidations of activities, functions and structures of biological samples such as cells, cell organelles, proteins, nucleic acids, phospholipids and the like at the micron level have been demanded concurrent with rapid advancement of investigations in the field of life science. Further, in connection with these elucidations, studies on possibility of the application of biological samples to various types of devices, starting with the application to electric elements, and studies on the application to screening techniques such as DNA chips, protein chips and the like have been extensively conducted. Examples of the various types of devices include biosensors, switching elements, bioreactors, hybrid artificial organs, neuro computers, DNA computers and the like.

Techniques to immobilize a biological sample in a desired minute region in a state where the function thereof is maintained can be a clue to development and popularization of technologies, in any of the aforementioned elucidations of biological samples at the micron level, device techniques or screening techniques in which the biological sample is used as an element.

For example, the present applicant filed an application in respect of an apparatus for measuring extracellular electric potential equipped with an electrode by which stable observation of the electrophysiological activity of a cell for a long time period is enabled without causing any damage to the cell or tissue (see, JP-A No. 6-78889 and JP-A No. 6-296595).

When a nerve cell or the like becomes active, change of the ionic permeability in the nerve cell is caused, and the ion concentrations inside and outside of the cell membrane are changed concurrently thereto. In the apparatus for measuring extracellular electric potential described above, observation of an activity of a nerve cell is enabled by immobilizing a cell on an electrode, and measuring the change of electric potential of the electrode caused upon the change of the ion concentration in the vicinity of the nerve cell. Therefore, also in the aforementioned apparatus for measuring extracellular electric potential, techniques for immobilizing a cell only onto the surface of an electrode have been desired. By certainly immobilizing a cell only onto the surface of an electrode, the electrophysiological activity of each cell can be detected, and in addition, detection of an interaction between cells is also permitted from the results of the electrophysiological measurement of multiple cells.

Immobilization of a biological sample in a desired region on a basal plate in a state where the function thereof is maintained is extremely difficult, and additional difficulty is involved when the desired region is minute. In particular, when a cell is employed for use in detecting its activity, and for use in which its activity is utilized, the cell is required to be immobilized as it is alive, which has been difficult because application of other method of immobilizing a chemical substance is not allowed.

Conventionally, as a method of immobilizing a cell, which is in a state where the activity thereof is maintained, in a desired region, a method of immobilizing a cell in a desired region has been known in which presence of a cell adhesive polymer is allowed in a region to which cell adhesion is intended, while presence of a polymer that is not adhesive to a cell is allowed in the remaining region, and thereby effecting the immobilization of a cell in the desired region (see, JP-A No. 7-51061). Further, a method in which a region for settling a cell is physically restricted by placing a metal mask with a through-hole on a basal plate (see, Yasuhiko Jimbo et al. Simultaneous Measurement of Intracellular Calcium and Electrical Activity from Patterned Neural Networks in Culture. IEEE Transaction on Biomedical Engineering Vol.40, No.8 pp.804-810, 1993). In addition, a method in which a cell is subjected to patterning by way of an electrostatic action using an electrified drum has been known (see, JP-B No. 2-245181).

However, such patterning of arrangement of a cell through the modification of a surface as the method of immobilizing a cell in a desired region by using a cell adhesive polymer and a polymer that is not adhesive to the cell results in unclear boundary region. Therefore, it is difficult to select a minute region to permit adhesion of a cell. Further, in the method of using a metal mask with a through-hole, there exists a problem of detachment of the cell upon removing the mask, which manipulation carried out by hand. Therefore, this method is also not satisfactory. Moreover, in the method of using an electrified drum, there exist drawbacks in connection with restriction of the usable basal plate only to any one of those which are suited for the shape of the drum, and also with requirement of an apparatus in a large scale.

As stated hereinabove, any method in the prior arts was not sufficient as a method of immobilizing a cell with high accuracy in a desired minute region.

### BRIEF SUMMARY OF THE INVENTION

The present invention was made taking into account of such a current state of art, and an object of the present invention is to provide a method of the immobilization in which a cell can be immobilized with high accuracy, in a state where the activity thereof is maintained, even onto a minute region, or a region that constitutes a complicated pattern.

In order to accomplish the object described above, the present invention involves a method of the immobilization of a cell in which a cell is immobilized in a desired region among the surface of a substrate, which comprises: the step (a) of forming a masking layer in a region except for the aforementioned desired region on the surface of the aforementioned substrate, the step (b) of immobilizing the aforementioned cell following the step (a) through bringing a solution containing the aforementioned cells into contact with the surface of the aforementioned substrate and the surface of the aforementioned masking layer, and the step (c) of adjusting the pH of the aforementioned solution to give the condition which permits separation of the aforementioned masking layer from the aforementioned substrate without loss of the activity of the aforementioned cell following the step (b).

In the method of the immobilization of a cell described above, the aforementioned masking layer is separated from the aforementioned substrate in the step (c). At the same time, the cells which had been immobilized on the surface of the aforementioned masking layer is also separated from the aforementioned substrate together with the aforementioned masking layer. Accordingly, the state in which only the cell immobilized in the desired region on the surface of the aforementioned substrate is immobilized is maintained. The cell is immobilized in a state in which its activity is kept, i.e., in a living state, in this occasion. Examples of the aforementioned solution which may be used include e.g., culture mediums, and physiological saline. Preferably, a culture medium is used because the cell can be proliferated concomitant with the immobilization of the cells, when a culture medium is used.

For example, the step (a) comprises the step of forming the aforementioned masking layer on the surface of the aforementioned substrate, and the step of removing the aforementioned masking layer in the aforementioned desired region. In this instance, it is desired that the aforementioned masking layer is formed from a masking material having photosensitivity, and the step of removing the aforementioned masking layer is the step of exposing either one of the aforementioned masking layer in the aforementioned desired region or in the region other than the aforementioned desired region, followed by development. When a masking layer is formed in such a step, the masking layer can be formed with high resolution even though it is in a complicated pattern or in a minute region.

When a masking material having photosensitivity is used, a masking material, which enables separation of the aforementioned masking layer from the substrate in the step (c) by, for example, elevating the pH of the aforementioned solution in the step (c) greater than the pH of the aforementioned solution in the step (b) can be used. Alternatively, a masking material, which enables separation of the aforementioned masking layer from the substrate in the step (c) by, for example, adjusting the aforementioned pH to be 7.9 or greater and 8.1 or less in the step (c) can be used.

In the step (c), the pH of the aforementioned solution can be adjusted by for example, adjusting the concentration of carbon dioxide in the ambient atmosphere of the aforementioned solution. Alternatively, the pH of the aforementioned culture medium can be adjusted by adding a pH adjusting agent to the aforementioned solution in the step (c).

It is preferred that the step of heating the aforementioned masking layer is included after the step (a) and before the step (b). In this instance, the aforementioned heating is conducted at a temperature higher than the boiling point of a detrimental constituent, which is included in the aforementioned masking layer, to the aforementioned cell. By carrying out such a treatment, the detrimental constituent, which is included in the masking layer, to the cell can be vaporized. In other words, loss of activity of the cell due to the detrimental constituent included in the masking layer can be prevented.

In the step (b), the aforementioned cell is preferably immobilized via an immobilization material, because immobilization of the cell can be facilitated and/or strengthened even though the surface of the aforementioned substrate consists of a material which involves difficulty in immobilizing a cell. The aforementioned immobilization material which may be used is for example, a material including a cell adhesive protein, a positively charged polymer, or a polymer having a strongly basic functional group.

The object as described above, other objects, characteristics, and advantages of the present invention will be apparent from the following detailed description of the preferred embodiments with reference to the accompanying drawings.

### BRIEF DESCRIPTION OF THE DRAWINGS

FIG. 1 is a flow chart showing a method of the immobilization of a cell according to the first Embodiment.

FIG. 2 is a drawing schematically showing a method of the immobilization of a cell according to the present invention.

FIG. 3 is a cross sectional drawing schematically showing the constitution of an integrated composite electrode according to the second Embodiment.

FIG. 4 is a cross sectional drawing viewed along the line A-A depicted in FIG. 3, schematically showing the constitution of the integrated composite electrode according to the second Embodiment.

FIG. 5 is a schematic drawing showing an apparatus for measuring extracellular electric potential according to the second Embodiment.

FIG. 6 is a top view showing a sensor part of a cell immobilization device according to the third Embodiment.

FIG. 7 is a cross sectional drawing schematically showing the constitution of a cell immobilization device according to the fourth Embodiment.

FIG. 8 is a cross sectional drawing viewed along the line B-B depicted in FIG. 7, schematically showing the constitution of a cell immobilization device according to the fourth Embodiment.

FIG. 9 is a top view showing a sensor part of a cell immobilization device according to the fifth Embodiment.

FIG. 10 is a drawing illustrating results of the measurement in Example 1.

FIG. 11 is a drawing illustrating results of the measurement in Example 2.

### DETAILED DESCRIPTION OF THE INVENTION

The present invention is explained in more detail below.

(First Embodiment)

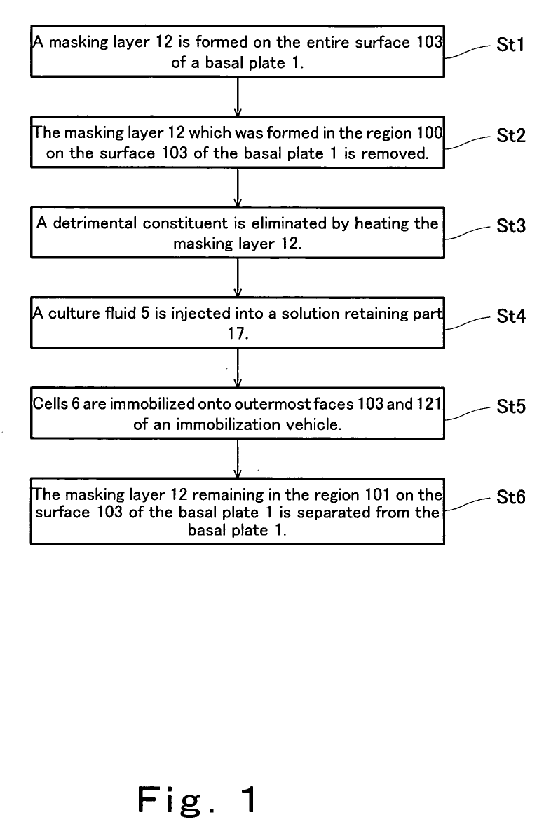

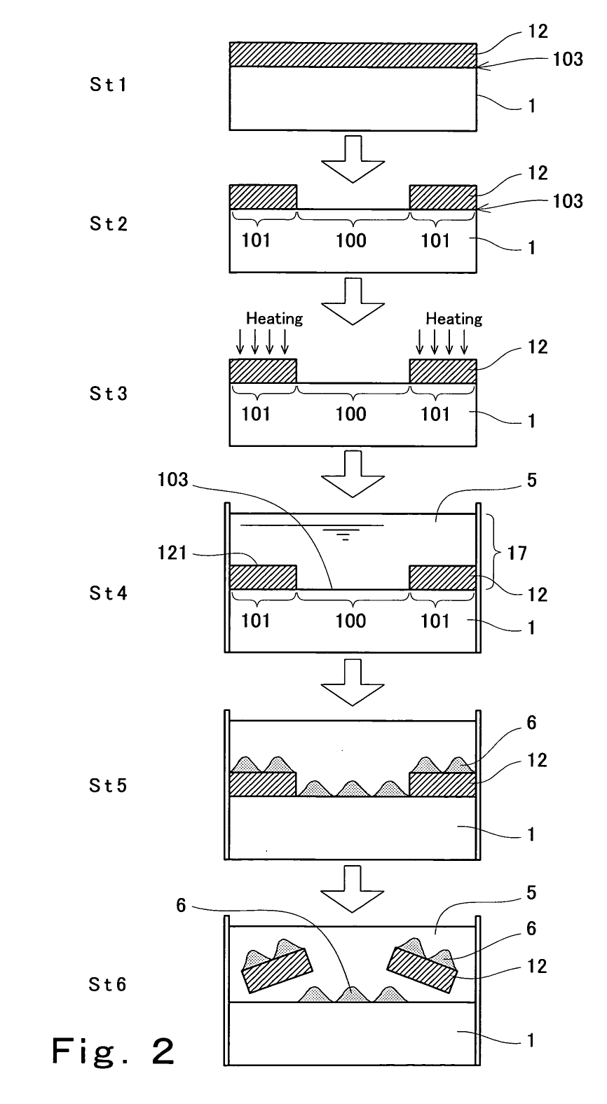

The method of the immobilization of a cell according to the first Embodiment is explained with reference to FIG. 1 and FIG. 2. In this Embodiment, a basal plate is used as a substrate for immobilizing a cell. FIG. 1 is a flow chart showing the method of the immobilization of a cell according to this Embodiment. FIG. 2 is a drawing schematically showing the method of the immobilization of a cell according to this Embodiment.

In the method of the immobilization of this Embodiment, a masking material is first applied on the surface 103 of a basal plate 1 to form a masking layer 12 (St1). Next, the masking layer 12 in an area corresponding to a region 100 on the surface 103 of the basal plate 1 is removed (St2). Thus, the masking layer 12 is formed in a region 101 except for a desired region 100 on the surface 103 of the basal plate 1. As the masking material, a photosensitive material can be suitably used. When a positive photosensitive material is used, the masking layer 12 only in the area corresponding to the region 100 is exposed, to the contrary, when a negative photosensitive material is used, the masking layer 12 only in the area corresponding to the region 101 is exposed, in St2. After the exposure, the masking layer 12 only in the area corresponding to the region 100 can be removed by development. The exposure and development in such a step can be conducted according to a conventional method. As the material for forming the masking layer 12, any one which permits separation of the mask layer 12 from the basal plate 1 by a treatment illustrated in St6 below may be used.

When a detrimental constituent to the cell is included in the masking material, a treatment of eliminating such a detrimental constituent is conducted following the formation of the masking layer 12. For example, when the detrimental constituent is vaporized by heating, the masking layer 12 is heated to a temperature higher than the boiling point of the detrimental constituent, thereby eliminating the detrimental constituent through vaporization (St3).

Next, after constructing a solution retaining part 17, the bottom face of the solution retaining part 17 is coated with an immobilization material, and then a culture medium 5 is injected into the solution retaining part 17 (St4). The solution retaining part 17 is constructed by providing a divider member 4 in a region having the outermost faces 103 and 121 of the immobilization vehicle, as a bottom face, which include the surface 103 of the basal plate 1 and the surface 121 of the masking layer 12 such that a solution can be retained in the region.

Then, cells 6 are seeded into the culture medium 5 to immobilize the cells 6 on the outermost faces 103 and 121 of the immobilization vehicle (St5). When the cells 6 are seeded into the culture medium 5, adhesive cells are spontaneously immobilized on the outermost faces 103 and 121 of the immobilization vehicle.

The immobilization material referred to herein is a material which facilitates and/or strengthens the immobilization of the cell 6. The immobilization material may be optionally selected depending on the type of the cell 6 to be immobilized. For example, a matrix material such as collagen, fibronectin, vitronectin, laminin or the like that is a cell adhesive protein is suitably used as the immobilization material. Alternatively, a positively charged polymer such as polyethyleneimine (PEI), polyornithine (PO), polylysine (PL) or the like, or a combined material of these materials is suitably used. Alternatively, a polymer having a strongly basic functional group such as a biguanide group, a carbamoylguanidide group or the like is suitably used. Specifically, such a polymer includes allylbiguanide-co-allylamine (PAB), allyl-N-carbamoylguanidino-co-allylamine (PAC).

Cells are negatively charged on their cell membrane in general, therefore, an electrostatic interaction is effected with the positively charged polymer or the polymer having a strongly basic functional group. Accordingly, immobilization of the cell 6 onto the outermost faces 103 and 121 of the immobilization vehicle can be facilitated and/or strengthened. The term “positively charged polymer” referred to herein is a polymer that is positively charged at the pH of equal to or less than the pKa value of the polymer.

After immobilizing the cells 6, the masking layer 12 in the area corresponding to the region 101 is separated from the basal plate 1 (St6). In the method of the separation, the pH of the culture medium 5 is adjusted, and left the fluid to stand for a predetermined time period. The pH of the culture medium 5 is set to be the pH which permits separation of the masking layer 12 from the basal plate 1 after leaving the fluid to stand for a predetermined time period. The aforementioned pH varies depending on the adhesive strength between the masking layer 12 and the basal plate 1 (dependent on the conditions of formation involving the material of the masking layer 12, the material of the basal plate 1, temperature of the treatment, and the like), therefore, the pH is selected to meet such conditions of formation. The aforementioned pH should be in the range not to result in loss of the activity of the cell 6. On the other hand, the pH of the culture medium 5 in St5 should be in the range not to result in separation of the masking layer 12. For example, the culture medium 5 may have the pH of approximately neutral (6.8 or greater and 7.8 or less) in St5, and the culture medium may have the pH of 7.9 or greater and 8.1 or less in St6.

The pH of the culture medium 5 in St6 can be adjusted by adjusting the concentration of carbon dioxide in the ambient atmosphere of the culture medium 5, or alternatively, it can be adjusted by adding a pH adjusting agent to the culture medium 5. The culture medium 5 in St5 is used as it is in St6, however, for example, the culture medium 5 in St5 may be changed into other solution in which the cell 6 can survive, and then separation of the aforementioned masking layer 12 may be permitted depending on the pH of the aforementioned solution. Examples of such a solution which may be used include other culture mediums and physiological saline.

In St6, the cells 6 which had been immobilized in the region other than the desired region 100 on the surface 103 of the basal plate 1 are consequently separated from above the basal plate 1 together with the masking layer 12. According to the method described hereinabove, immobilization of the cell 6 only in the desired region 100 on the basal plate 1 is enabled. After the separation of the masking layer 12 from the basal plate 1, thus separated masking layer 12 is removed from the culture medium 5.

As the masking material which forms the masking layer 12, a photosensitive material is preferably used. More preferably, a material which can be separated from the substrate by immersion in an alkaline solution, and which allows for elimination of the detrimental constituent to the cell by heating the same may be used. For example, a photosensitive material containing a polyimide resin can be used, and examples of such a material include CRC-8300 (trade name, manufactured by Sumitomo Chemical Co., Ltd.), RN-901 (trade name, Nissan Chemical Industries, Ltd.) and the like.

Through the use of a photosensitive material as a masking material, pattern formation by photo etching can be utilized in the step of forming the masking layer 12, thereby making it possible to immobilize the cell 6 onto a minute and complicated region.

As the basal plate 1, an arbitrary one can be used depending on the intended use of the immobilized cell 6. For example, when the immobilized cell 6 is used as a subject of an electrophysiological measurement, a basal plate with an electrode and a wiring formed thereto may be used (demonstrated in second to fifth Embodiments). In addition, when the immobilized cell is to be a subject of microscopic observation, a transparent basal plate may be used.

As the culture medium 5, an arbitrary culture medium may be selected depending on the type of the cell 6 that is a subject of the immobilization. For example, Eagle's basal medium (BME), minimum essential medium (EMEM), Dulbecco's modified Eagle's medium (DMEM), Joklik modified medium (joklik), 199 medium (199), RPMI-1640 (1640), Ham's F-10 medium (F-10), Ham's F-12 medium (F-12), Ham's F12K medium (F-12K), Leibowitz L-15 medium (L-15), McCoy's 5A medium (McCoy5A), NCTC135 medium (135), William's Emedium (William), Waymouth MB752/1 medium (Waymouth), CMRL-1066 (1066), Iscove's modified Dulbecco's modified medium (Iscove) or the like may be used. Moreover, a variety of nutrients, growth factors, antibiotics and the like may be further added to the medium as described above for use as the culture medium 5. In St5, a culture medium 5 without having any action to permit separation of the masking layer 12 from the basal plate 1 is selected until the immobilization of the cell 6 is completed.

(Second Embodiment)

This Embodiment relates to a method of the immobilization in which a cell is immobilized on an electrode of a cell immobilization device. The cell immobilization device described above is one of the constitutive elements of an apparatus for measuring extracellular electric potential used for detecting an electrical signal resulting from the electrophysiological change of a cell. The constitution of the apparatus for measuring extracellular electric potential equipped with the cell immobilization device used in this Embodiment is explained below.

[Constitution of a Cell Immobilization Device]

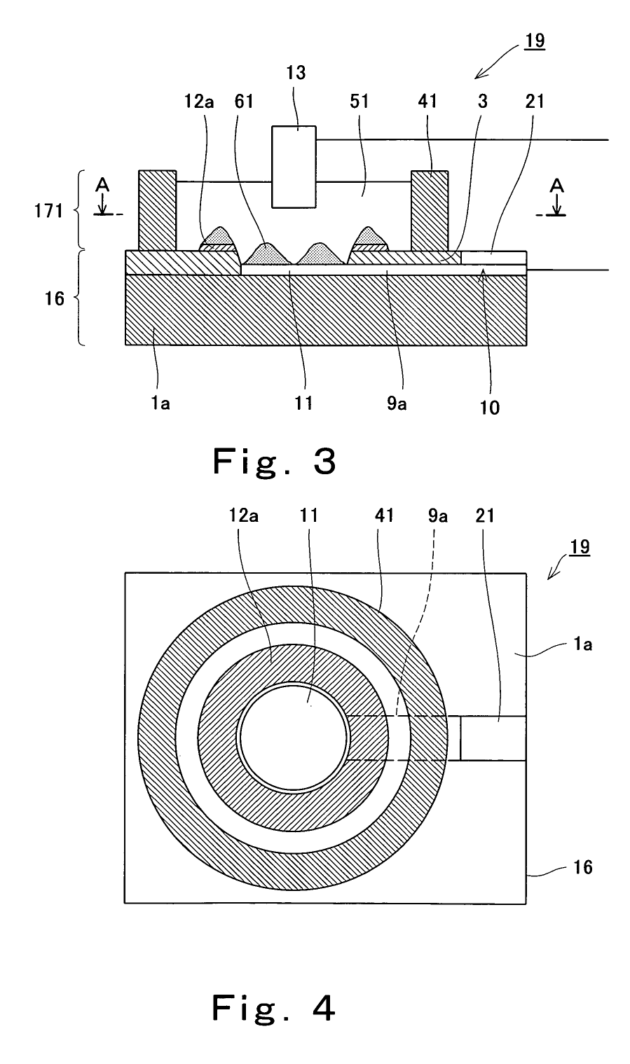

FIG. 3 is a cross sectional drawing schematically showing the constitution of a cell immobilization device 19 that constitutes an apparatus for measuring extracellular electric potential of this Embodiment. FIG. 4 is a cross sectional drawing viewed along the line A-A depicted in FIG. 3. FIG. 3 illustrates a state in which cells 61 are immobilized on the cell immobilization device 19 (i.e., the state in St5 shown in FIG. 2), and FIG. 4 illustrates a state prior to the immobilization of the cells 61 (i.e., the state in St4 shown in FIG. 2). Moreover, in FIG. 4, a lead wire 9a formed on the back face of a sensor part 16 is illustrated by a dashed line.

The cell immobilization device 19 comprises a sensor part 16 equipped with an electrode 11, and a solution retaining part 171. The sensor part 16 comprises an electrode 11 and a basal plate 1a equipped with a lead wire 9a that connects to the electrode 11. The lead wire 9a is coated with an insulating layer 3 on its upper face except for the external connection part 10. The upper face of the external connection part 10 of the lead wire 9a is coated with a coating layer 21. For the coating layer 21, an electric conductive material that has potent resistance to the surrounding atmosphere is selected depending on such a surrounding atmosphere to which the external connection part 10 is exposed. Durability of the external connection part 10 is improved by coating with the coating layer 21, however, it may not be necessarily coated by the coating layer 21.

Top face shape of the electrode 11 is preferably circular or square, e.g., with the diameter or the length of one side in the range of approximately 1 μm to 2000 μm. When size of the electrode is greater than the subject cell to be measured, an electrical signal derived from an electrophysiological activity of multiple cells can be detected with one electrode.

The cell immobilization device 19 is equipped with a solution retaining part 171 on the sensor part 16, for use in culturing the cell. The solution retaining part 171 comprises a cylindrical divider member 41 provided on the sensor part 16, an inner region of the divider member 41, and a reference electrode 13 provided within the inner region. The divider member 41 is provided after the step of the formation in which a masking layer 12a is formed to present a desired pattern in one of the steps of immobilizing the cell described below.

The reference electrode 13 has only to be immersed in a cell culture medium 51 during the measurement, and it may be previously fixed within the solution retaining part 171, or may be placed and fixed in the culture medium 51 upon the measurement. For example, the reference electrode 13 may be mounted on the inside wall of the divider member 41, although not shown in the Figure.

Examples of the basal plate which is preferably used as the basal plate 1a include those formed with a semiconductor material typified by single crystal silicon, amorphous silicon, silicon carbide, silicon dioxide, silicon nitride and the like; a composite material of these semiconductor materials typified by a silicon•on•insulator (SOI) and the like; an inorganic insulating material selected from the group consisting of glass, quartz glass, alumina, sapphire, forsterite, silicon carbide, silicon dioxide, and silicon nitride; and an organic material selected from the group consisting of polyethylene, ethylene, polypropylene, polyisobutylene, polyethylene terephthalate (PET), unsaturated polyester, a fluorocarbon resin, polyvinyl chloride, polychlorinated vinylidene, polyvinyl acetate, polyvinyl alcohol, polyvinyl acetal, an acrylic resin, polyacrylonitrile, polystyrene, an acetal resin, polycarbonate (PC), polyamide, a phenol resin, a urea resin, an epoxy resin, a melamine resin, a styrene•acrylonitrile copolymer, an acrylonitrile•butadiene styrene copolymer, a silicon resin, polyphenylene oxide and polysulfone. More preferably, a basal plate that is formed with single crystal silicon, SOI, PET, or PC may be used.

The electrode material which is preferably used for forming the electrode 11 may be a metal material selected from the group consisting of platinum black, platinum, gold, palladium, rhodium, silver, and tungsten; or a metal oxide material selected from the group consisting of titanium oxide, tin oxide, manganese oxide, lead oxide, and indium tin oxide (ITO). The electrode 11 may be formed using one material selected from these materials, or multiple kinds of materials may be deposited into e.g., layers, to form the electrode 11. The upper face of the electrode 11 may be further coated with an electric conductive polymer or a unimolecular membrane. Also as a lead wire material which forms the lead wire 9a that is connected to the electrode 11, a similar material to that for the aforementioned electrode material can be suitably used.

The electrical signal from the electrode 11 is measured with the electric potential of the reference electrode 13 as a standard. In general, the reference electrode 13 has its surface area of equal to or greater than the surface area of the electrode 11, and preferably, greater than the surface area of the electrode 11. The reference electrode 13 is preferably made from a material such as gold, platinum, silver-silver chloride or the like, however, size and shape thereof can be determined ad libitum.

The divider member 41 can be made from for example, acryl. The divider member 41 is acceptable as long as it is constituted such that it can retain a cell culture medium 51 in its inner region of which bottom face agrees with the upper face of the sensor part 16 including the upper face of the electrode 11, but the shape thereof is not limited to cylindrical.

[Method of Producing a Cell Immobilization Device]

An example of the method of producing the cell immobilization device 19 is illustrated. A desired pattern is formed having multiple sets, the set being an electrode 11 and a corresponding lead wire 9 as one set, by vapor deposition of an electrode material on a basal plate 1a first, followed by use of a photoresist to execute the etching. Thereafter, the upper face of the lead wire 9a except for an external connection part 10 is coated with an insulating layer 3. Further, the upper face of the external connection part 10 of the lead wire 9a is coated with a coating layer 21. Thereafter, the basal plate 1a is cut out into small pieces with a predetermined angle. Thus, one small piece is defined as a sensor part 16. One small piece is produced such that a set of the electrode 11 and the lead wire 9a are formed therein. Pattern of the electrode 11 may be formed by a lift off method or a masking method in which vapor deposition is conducted through a stencil mask having the aforementioned pattern previously formed.

[Method of the Immobilization of Cells]

A method of immobilizing cells on the electrode 11 of the sensor part 16 described above using the method of the present invention is explained. First, a masking layer 12a is formed in a region involving at least a surrounding region of the electrode 11 but excluding the electrode 11 among the outermost surface of the sensor part. The masking layer 12a is formed not to locate in a region which is going to be provided with a divider member 41 in the later step.

The masking material which may be suitably used for forming the masking layer 12a is similar to that described in the first Embodiment. The masking material should be a different material from that for use in the insulating layer 3, and in addition, a material which can be separated without removing the insulating layer 3 under the condition for the separation is used.

Through the use of a photosensitive material for the masking layer 12a, the masking layer 12a can be formed in the region other than the upper face of the electrode 11 utilizing pattern formation by photo etching. After forming the masking layer 12a in a wide region including the electrode 11 on the upper face of the sensor part 16, the upper face of the electrode 11 can be exposed through removing the masking layer 12a on the part of the electrode 11 on the sensor part 16 by photo etching.

After the step of forming the masking layer 12a, the divider member 41 is provided on the sensor part 16 to surround the electrode 11, as described above, thereby constructing the solution retaining part 171.

Thereafter, an immobilization material is applied on the region within the solution retaining part 171 among the uppermost face of the sensor part 16. The immobilization material which may be used is a material that does not cause degeneration of the cell membrane of the cell. Therefore, any material leading to immobilization of a cell by causing a crosslinking reaction between the immobilization material and the cell membrane is not used, for the purpose of detecting an electrical signal derived from the electrophysiological change of a cell which is approximate to a state in a living body. Any immobilization material presented in the first Embodiment may be suitably used.

Immobilization of the immobilization material is conducted by exposing a solution which contains an immobilization material dissolved therein to give a predetermined concentration on the electrode 11, removing the same from the upper face of the electrode 11 after lapse of a predetermined time period, washing the upper face of the electrode at least once, and drying. Also, an immobilization material may be immobilized on the electrode 11 by spotting a solution which contains an immobilization material dissolved therein on the electrode 11. When a matrix material such as a cell adhesive protein is used as an immobilization material, the matrix material is applied on the upper face of the electrode 11 by a known method.

Next, a culture medium 51 is charged to fill in the solution retaining part 171. The culture medium as presented in the first Embodiment is suitably used.

Then, desired cells 61 are seeded into the culture medium 51. Concomitantly to the progress of culture of the cells 61, the adhesive cells are immobilized on the uppermost face of the sensor part 16 via the immobilization material. After immobilizing the cells, the culture medium 51 is conditioned to permit separation of the masking layer 12a, and left to stand for a while. Accordingly, the masking layer 12a is spontaneously separated from the upper face of the sensor part 16. Such a step enables the immobilization of the cells 61 only onto the upper face of the electrode 11.

Measurement of an electrical signal resulting from an electrophysiological change of the cells 61 is initiated in a state in which the cells 61 are immobilized on the uppermost face of the electrode 11. Measurement of the electrical signal refers to the measurement of the difference of electric potential between the electrode 11 and the reference electrode 13, on the basis of the electrical signals detected from a pair of electrodes 11 and 13. In the cell, ion permeability of the cell membrane is changed corresponding to the change of the activity of its ion channels, and the ion concentration inside and outside of the cell membrane is changed accompanied by such a change of ion permeability. In other words, gradient of the ion concentration inside and outside of the cell membrane is changed. Difference of electric potential between the electrode 11 and the reference electrode 13 is changed such that alteration of the gradient of the ion concentration is counter balanced. Therefore, by measuring the aforementioned difference of electric potential, an electrophysiological change of the cell can be indirectly detected. The aforementioned difference of electric potential can be measured by for example, using the apparatus for measuring extracellular electric potential described below.

[Constitution of an Apparatus for Measuring Extracellular Electric Potential]

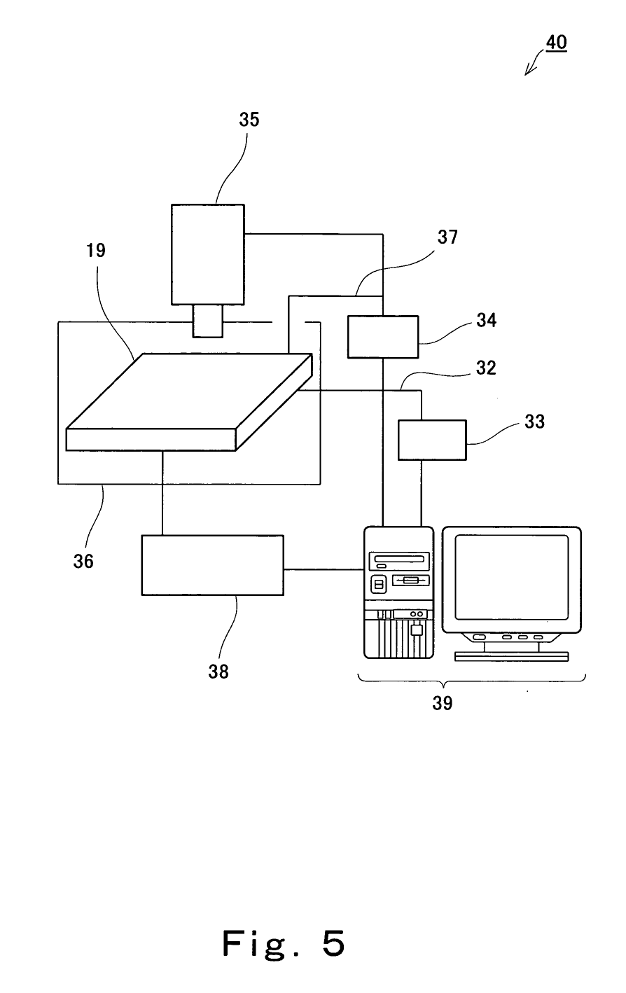

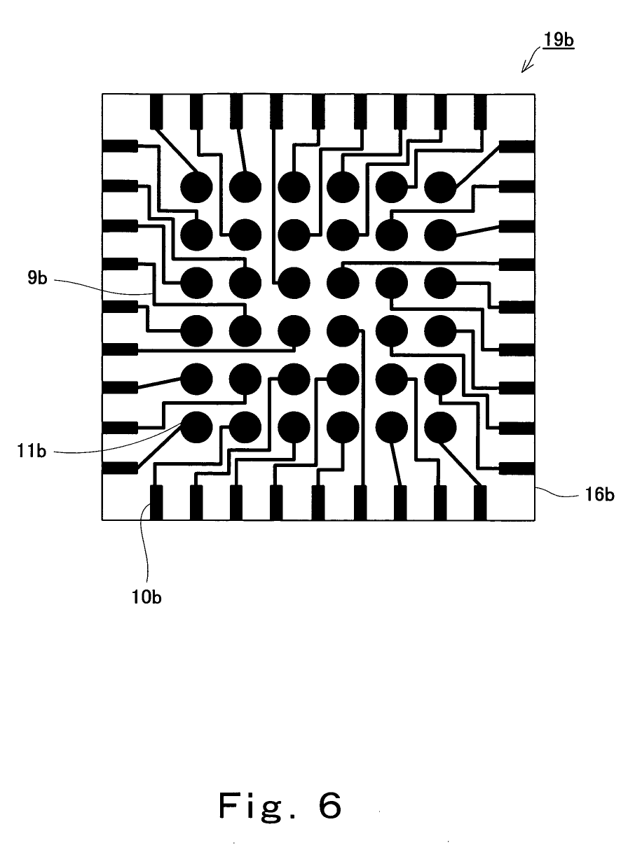

FIG. 5 is a schematic drawing showing an apparatus for measuring extracellular electric potential according to this Embodiment. The apparatus for measuring extracellular electric potential 40 comprises a controlling part 39, a signal amplification part 33 connected thereto, a stimulatory signal imparting part 34 and a solution driving part 38, an imaging part 35, and a mounting part 36.

The cell immobilization device 19 is mounted to the mounting part 36. The mounting part 36 has functions to keep the mounted cell immobilization device 19 at a predetermined temperature, gas concentration and humidity. The controlling part 39 detects and records the difference of electric potential between the electrodes 11 and 13 of the cell immobilization device 19, on the basis of the signal entered from the signal amplification part 33. In addition, the controlling part 39 controls the stimulatory signal imparting part 34 on the basis of the predetermined stimulating condition. The stimulatory signal imparting part 34 is equipped with a D/A transducer, and electrical stimulation is applied to the cell on the cell immobilization device 19 via the transducer and a line 37. The electrical signal from the cell immobilization device 19 is lead out to the signal amplification device 33 via the line 32. In the signal amplification device 33, the electrical signal is amplified, subjected to the limitation of the frequency band, and entered into the controlling part 39 via the A/D transducer.

The solution driving part 38 has functions to discharge the culture medium 51 retained within the solution retaining part 17 of the cell immobilization device 19, or to inject the culture medium 51 into the solution retaining part 171. It is driven by the controlling part 39 as needed. Using the imaging part 35, the electrode 11 on the cell immobilization device 19 can be put into an image or observed. Further, the stimulatory signal imparting part 34 may be constituted such that the output stimulatory signal is selected, on the basis of imaging data from the imaging part 35.

The apparatus for measuring extracellular electric potential 40 imparts a stimulatory signal from the stimulatory signal imparting part 34 to the cells 61, and can detect the electrophysiological change of the cells 6 corresponding to the response thereof. Alternatively, it is also possible to detect the electrophysiological change which is spontaneously generated in the cells, without imparting a stimulatory signal.

(Third Embodiment)

This Embodiment concerns a cell immobilization device having a different constitution from that in the second Embodiment. FIG. 6 is a drawing showing a upper face of a sensor part 16b of the cell immobilization device 19b according to the third Embodiment. The cell immobilization device 19b is constituted such that electrodes 11b are arranged at each intersecting point of a lattice having 6 rows and 6 columns. The cell immobilization device 19 of the second Embodiment has the constitution having only one electrode 11 formed on the sensor part 16, however, the cell immobilization device 19b of this Embodiment is constituted to have multiple electrodes 11b and corresponding lead wires 9b formed on one sensor part 16b.

Although FIG. 6 illustrates with a solution retaining part omitted therefrom, the divider member (having a similar constitution to the divider member 41 of the second Embodiment) that constitutes the solution retaining part may be provided either per every one electrode 11b, or per a set of multiple electrodes 11b. The constitution provided with the divider member per every one electrode 11b is useful, for example, in the measurement of responsiveness to a drug of the cell immobilized on each electrode 11b, whilst the constitution provided with the divider member per a set of multiple electrodes 11b is useful, for example, in carrying out the analysis of a network because a network can be formed among nerve cells immobilized on each electrode 11b.

Also in the instance where the sensor part 16b shown in FIG. 6 is used, the method of immobilizing cells described above can be employed.

Also in this Embodiment, immobilization of the cells only onto the electrodes 11b is enabled employing the method of immobilizing cells as presented in the second Embodiment.

(Fourth Embodiment)

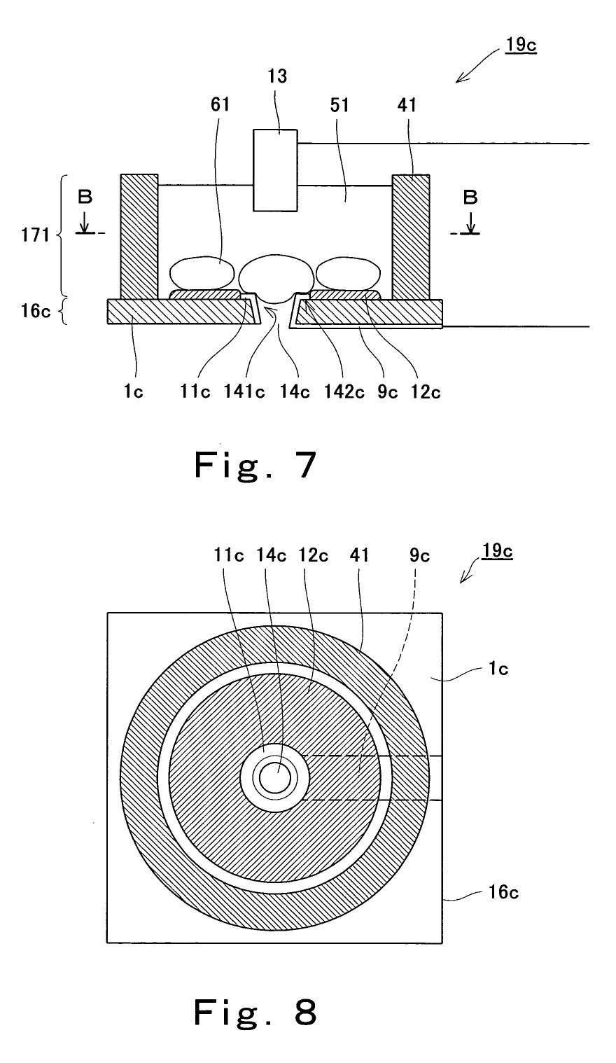

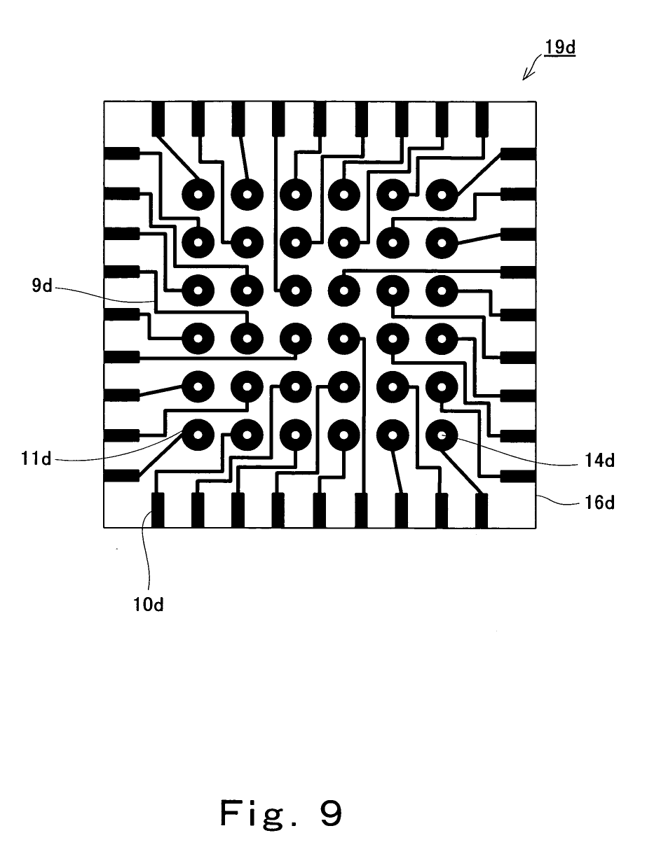

This Embodiment concerns a cell immobilization device having a different constitution from those in the second and the third Embodiments. FIG. 7 is a cross sectional drawing schematically showing the constitution of the cell immobilization device 19c according to this Embodiment. FIG. 8 is a cross sectional drawing viewed along the line B-B depicted in FIG. 7. FIG. 7 illustrates a state in which cells 61 are immobilized on the cell immobilization device 19c (i.e., the state in St5 shown in FIG. 2), and FIG. 8 illustrates a state prior to the immobilization of the cells 61 (i.e., the state in St4 shown in FIG. 2). Moreover, in FIG. 8, a lead wire 9c formed on the lower face of a sensor part 16c is illustrated by a dashed line.

Because the cell immobilization device 19c of this Embodiment has a different constitution in only the sensor part from the cell immobilization device 19 of the second Embodiment, the explanation of other constitutions except for the sensor part is omitted through assigning the identical number.

A basal plate 1c has a through-hole 14c. An electrode 11c is formed on the hole wall surface 141c and the marginal edge 142c of the hole opening of the through-hole 14c. The electrode 11c is formed by making an electrode material adhered on the hole wall surface 141c and the marginal edge 142c of the hole opening of the through-hole 14 using a vacuum vapor deposition method or a sputtering method.

On the lower face of the sensor part 16b is formed a lead wire 9c such that it connects to the electrode 11c. Hence, it is not required that the sensor part 16c and the divider member 41 are constituted separately, but they may be integrally formed. The lead wire 9c may be constituted such that it is formed on the upper face of the sensor part 16c.

The through-hole 14c has a truncated cone shape having the upper face opening that is greater than the lower face opening. A part of cells 61 is captured at the through-hole 14c, and the cells 61 are retained on the sensor part 16c with close contact thereto. Since the through-hole 14c has a truncated cone shape, wide contact area with the cell 61 can be secured. However, the shape of the through-hole 14c is not limited to such a truncated cone shape, but any shape capable of capturing a part of cells 61 is acceptable. Size of the through-hole 14c which can be employed may be an arbitrary size depending on the subject cell 61 to be captured. For example, the diameter of the upper face opening of the sensor part 16c is in the range of 5 μm or greater and 100 μm or less, whilst the diameter of the lower face opening is in the range of 1 μm or greater and 10 μm or less. Suitably, an illustrative example may be that the diameter of the opening on the upper face is approximately 20 μm, whilst the diameter of the opening on the lower face is in approximately 5 μm, and the depth is approximately 15 μm, when the major axis of the cell which is the subject to be immobilized is approximately 30 μm.

Method of forming a through-hole 14c may vary depending on the material of the basal plate 1c, however, when the basal plate 1c consists of PET, it can be formed using, for example, an excimer laser. In addition, when the basal plate 1b is a Si wafer, it can be formed by, for example, etching.

Moreover, a constitution is permitted in which an aspiration means is provided capable of aspirating the cell 61 from beneath the through-hole 14c. Such a constitution enables capturing of the cell 61 at the through-hole 14c to be more strengthened, and thus the cell can be captured at the through-hole 14c even though the cell tends to float.

Any of the material of a basal plate for forming the basal plate 1c, the electrode material for forming the electrode 11c, and the masking material for use in forming the masking layer 12c in the step of immobilizing cells, which can be used, may be the material as presented in the first Embodiment.

Also in this Embodiment, the cells can be immobilized onto a desired position employing the method of immobilizing cells as presented in the second Embodiment. The method of the immobilization according to the present invention is responsible for the immobilization of cells 61 onto the electrode 11c as well as the through-hole 14c, and in addition, it prevents the cells 61 from immobilizing onto a region other than the desired region.

(Fifth Embodiment)

This Embodiment concerns a cell immobilization device having a different constitution from those in the second, the third and the fourth Embodiments. FIG. 9 is a top view showing the sensor part 16d of the cell immobilization device 19d according to this Embodiment. A lead wire 9d is not appeared on the upper face because it is formed on the lower face of the sensor part 16d, however, in FIG. 9, the lead wire 9d is shown on the upper face for the sake of convenience. The cell immobilization device 19d is constituted such that electrodes 11d are arranged at each intersecting point of a lattice having 6 rows and 6 columns. The cell immobilization device 19c of the fourth Embodiment has the constitution including only one electrode 11c formed on the sensor part 16c, however, the cell immobilization device 19d of this Embodiment is constituted to have multiple electrodes lid and corresponding lead wires 9d formed on one sensor part 16d.

Although FIG. 9 illustrates with a solution retaining part omitted therefrom, the divider member (having a similar constitution to the divider member 4 of the second Embodiment) that constitutes the solution retaining part may be provided either per every one electrode 11d, or per a set of multiple electrodes lid. The constitution provided with the divider member per every one electrode 11d is useful, for example, in the measurement of responsiveness to a drug of the cell immobilized on each electrode 11d, whilst the constitution provided with the divider member per a set of multiple electrodes lid is useful, for example, in carrying out the analysis of a network because a network can be formed among nerve cells immobilized on each electrode 11d. A lead wire 9d is not formed on the upper face of the sensor part 16d, therefore, the sensor part 16d and the divider member are not necessarily constituted separately, but it is possible to give an integrated formation.

Also in this Embodiment, the cell can be immobilized onto the electrode 11d employing the method of immobilizing cells as presented in the second Embodiment. The method of the immobilization according to the present invention is responsible for the immobilization of cells 61 onto the electrode 11d as well as the through-hole 14d, and in addition, it prevents the cells 61 from immobilizing onto a region other than the desired region.

### EXAMPLES

Examples of the present invention are demonstrated below. These Examples do not limit the present invention.

### Example 1

Example 1 is an example relating to the first Embodiment. An SOI wafer was used as a basal plate 1; RN901 was used as a material for a masking layer 12; PEI and collagen (Sigma P-4511) were used as an immobilization material; and smooth muscle cells derived from rat aorta VSMCs A-10 (ATCC No. CRL-1476) were used as cells 6 which are a subject to be immobilized.

First, the SOI wafer in 4 inches was subjected to dehydrating baking at 110° C. for 5 minutes. Thereafter, RN901 was spin coated at 300 rpm for 5 seconds, and at 3500 rpm for 30 seconds. After prebaking the spin coated wafer at 80° C. for 10 minutes, a circular pattern having the diameter of 100 μm was exposed and developed. After baking the wafer post development at 150° C. for 5 minutes, it was further baked at 170° C. for 60 minutes and at 350° C. for 60 minutes. In such baking steps, detrimental constituents having the boiling point of approximately 350° C. or less were eliminated.

After the baking, the wafer with the patterning of RN901 was washed with 70% EtOH. A divider member for forming a solution retaining part was provided on the wafer, and was coated with 0.1% by weight PEI for 3 hours, followed by sufficient rinsing with sterile water. Then, collagen (Sigma P-4511) which is a cell adhesive protein was coated on the wafer by a predetermined method at 37° C. for 30 minutes.

Thereafter, the solution retaining part was filled with a culture medium, and smooth muscle cells derived from rat aorta VSMCs A-10 (ATCC No. CRL-1476) were seeded on the wafer. The culture medium employed was HEPES buffered DMEM+10% by weight FBS. Then, 4 days culture was conducted in an atmosphere of 37° C. and the concentration of CO₂ of 5% by weight. Thereafter, the wafer was removed from the atmosphere of 37° C. and the concentration of CO₂ of 5% by weight, and left to stand to keep the temperature of 37° C., in an atmosphere of low CO₂ concentration (concentration of CO₂ Of 0.4% by weight or less). After four days, the masking layer 12 was detached from the wafer. The detached masking layer was carefully removed, and thus a circular pattern with cells immobilized thereon was observed only within the region of the diameter of 100 μm. Staining with trypan blue confirmed that the immobilized cells were alive.

Next, electric potential derived from the activity of the cells which were confirmed to be alive was detected with a patch clamp. For the measurement with a patch clamp, Current-Clamp mode with the holding current of 0 ampere (A) was employed. Upon the measurement of action potential with a patch clamp, the culture medium was replaced with a measurement fluid. The measurement fluid for use was Tyrode's solution including HEPES having a buffering action with a Ca ion. The solution was adjusted to give the Ca ion concentration of 2 mM, pH of 7.4, and osmotic pressure of 273 mOsm. As the inner solution to be filled in the patch pipette, an aqueous solution containing potassium chloride, EGTA, Ca ion, HEPES having a buffering action was prepared. The inner solution was adjusted to be 10 mM of HEPES and the pH of 7.2. EGTA chelates the Ca ion.

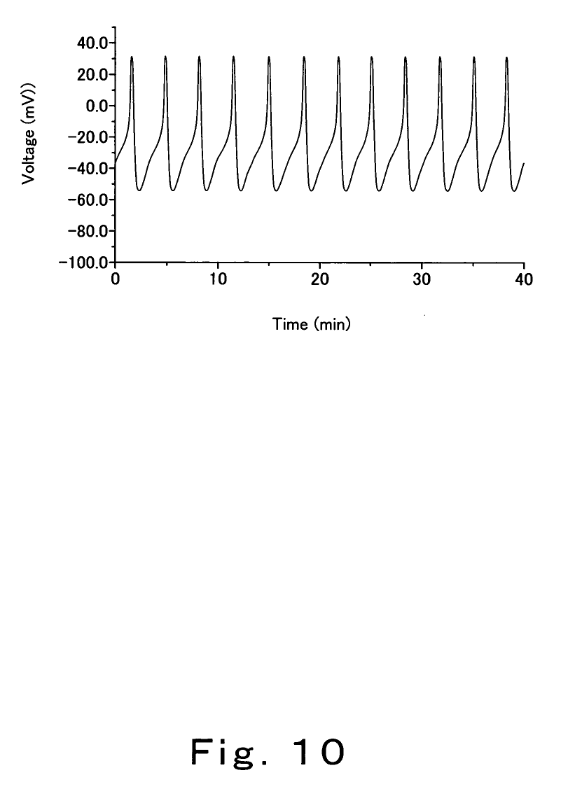

FIG. 10 is a drawing illustrating the change of electric potential detected from the cells. The horizontal axis represents time, and the longitudinal axis represents voltage (i.e., intensity of the electric potential which represents a cellular activity). As is clear from FIG. 10, resting potential of the cell is approximately 50 mV, and the cell continuously generated the change of electric potential with amplitude of approximately 80 mVp-p in a periodic manner. On the other hand, when the measurement fluid was replaced with a measurement fluid without including Ca ion, observation of periodic change of the electric potential was lost, although not shown in the Figure. Accordingly, the periodic change of the electric potential observed in FIG. 10 suggests that each ion channel that is present on the cell membrane and is involved in Ca ion permeability opens/shuts through sensitization with electric potential within the cell, thereby adjusting the electric potential within the cell to effect the cellular activity. Thus, it was confirmed that the immobilized cells kept having their activity.

On the other hand, in Comparative Example 1-1, similar treatment was carried out until the cells 6 were immobilized on a wafer, and thereafter, the wafer was left to stand in an atmosphere which is a similar atmosphere to that in the step of the cell culture (37° C., CO₂ concentration of 5% by weight). The masking layer 12 was not detached after the lapse of 8 days.

In Example 1, measurement of the pH of the culture medium 5 when the masking layer 12 was detached indicated the value of 7.9. During the culture, the pH of the culture medium 5 was kept neutral, approximately at 7.4, because it was in an atmosphere of the CO₂ concentration of 5% by weight. From the results of Example 1 and Comparative Example 1-1, it is revealed that the masking layer 12 was detached in Example 1, due to the change of the pH value of the culture medium 5 into an alkaline range, because the culture medium 5 was left to stand in the air.

A similar experiment was conducted using CRC8300 instead of RN901 described above, as the masking material. In this instance, the masking layer 12 was detached from the wafer after 7 days since the wafer was left to stand in an atmosphere of low CO₂ concentration (concentration of CO₂ of 0.4% by weight or less).

In Comparative Example 1-2, an experiment was carried out similarly to Example 1 except that only the condition of baking step was altered. The baking step in Comparative Example 1-2 was: baking of the wafer post development at 150° C. for 5 minutes, followed by additional baking at 170° C. for 60 minutes and at 320° C. for 30 minutes.

According to Comparative Example 1-2, immobilization of the cell on the wafer was not ascertained. From the results in Comparative Example 1-2, it is speculated that detrimental constituents to the cell were not eliminated from the masking layer 12 in the baking step of Comparative Example 1-2.

### Example 2

Example 2 is an example relating to the third Embodiment.

(Production of the Sensor Part Shown in FIG. 6)

First, a photoresist is spin coated on an Si wafer in 4 inches. Multiple patterns with circular electrodes 11b having the diameter of 5 μm that are positioned at each intersecting point of a lattice having 6 rows and 6 columns, and with the center distance of the electrodes 11b being 20 μm, and the pattern having lead wires 9b that radially spread from each electrode 11b, were exposed and developed on a predetermined position of an Si wafer. A gold thin film was deposited on the entire face of the wafer by a vacuum vapor deposition method. Thereafter, patterning of gold electrodes was completed on the Si wafer. Then, only the lead wire 9b parts were coated with a negative photosensitive polyimide resin UR-8300 (manufactured by Toray Industries, Inc.) that is an insulating material which requires a dedicated developing fluid, followed by dice cutting to cut out small pieces of 40 mm-square. Each small piece was constituted such that circular electrodes 11b having the diameter of 5 μm arranged on 6 rows and 6 column were exposed in its center, and lead wires 9b lead from the electrodes 11b were communicated to distributing drawing terminals arranged all around thereof. The small piece produced in such a manner was defined as a sensor part 16b.

(Immobilization of the Cell)

On the upper face of the sensor part 16b produced as described above was spin coated with CRC8300 at 800 rpm for 10 seconds, and at 4000 rpm for 30 seconds. After prebaking the spin coated sensor part at 110° C. for 6 minutes, a circular pattern with the diameter of 5 μm corresponding to the electrodes 11b (6 rows and 6 columns) was exposed and developed. Accordingly, the masking layer was formed in the region other than the upper face of the electrodes 11b. After baking the wafer post development at 150° C. for 5 minutes, it was further baked at 300° C. for 40 minutes, and at 450° C. for 40 minutes.

Thereafter, the upper face of the sensor part 16b with the patterning of CRC8300 was washed with 70% EtOH. A divider member that constitutes the solution retaining part was provided on the upper face of the sensor part 16b such that all electrodes 11a are included in the divider member, and then the solution retaining part was filled with a culture medium. Next, thereto were seeded nerve cells prepared using a known method to persons skilled in the art from cerebral cortex of a fetal rat on day 17. Density of seeding on the sensor part 16b was 5×10⁴ cells/mL. Following the seeding, after culture in an atmosphere of the temperature of 37° C. and the concentration of CO₂ of 5% by weight for 5 hours, a tetramethylammonium hydroxide (TMAH) solution as a pH adjusting agent was injected into the culture medium. The masking layer was thereby detached from the sensor part 16b. The detached masking layer was carefully removed, and then the cells were cultured for two weeks. Accordingly, it was confirmed that a network was reconstructed among nerve cells on the electrodes.

The nerve cells then formed clusters consisting of 5 to 10 cells, and a network was constituted among the clusters. Confirmation of the reconstitution of the network was conducted by microscopic observation.

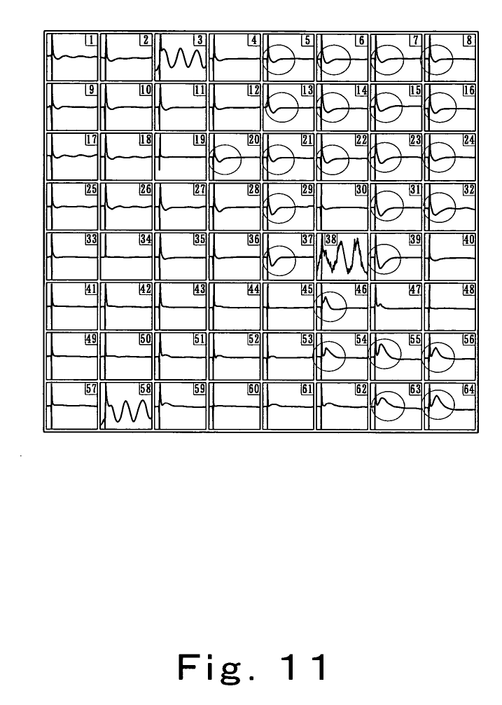

Bipolar constant current stimulation of 50 μA for 100 μsec was applied to one cell in one cluster on the reconstituted nerve cell network, and the transmission of the stimuli was measured from remaining 63 channels. The results are shown in FIG. 11. FIG. 11 shows the computer screen, which was printed out, displaying for each channel. The horizontal axis represents time, and the longitudinal axis represents voltage (i.e., intensity of the electric potential which represents a cellular activity) for each channel. The electrode to which the stimuli were applied is channel 38. As is clear from FIG. 11, cooperative transmission of the signal could be observed in the circled channels, i.e., 24 channels among 63 channels.

On the other hand, in Comparative Example 2, similar treatment to Example 2 was carried out until the cells were immobilized on a wafer, and thereafter, the wafer was left to stand in an atmosphere that is a similar atmosphere in the step of the cell culture (37° C., CO₂ concentration of 5% by weight). The masking layer 12 was not detached after the lapse of 8 days.

UR-8300 used for the insulating layer was not detached under the condition in which the masking layer described above was detached. Moreover, CRC8300 contains y-butyrolactone having the boiling point of 203° C. as a detrimental constituent, however, such a detrimental constituent was vaporized and eliminated on behalf of carrying out the baking at a temperature higher than 203° C. Therefore, such a detrimental constituent did not affect the cell.

In Example 2, measurement of the pH of the culture medium when the masking layer was detached indicated the value of 8.1. During the culture, the pH of the culture medium was kept neutral, approximately at 7.4, because it was in an atmosphere of the CO₂ concentration of 5% by weight. From the results of Example 2 and Comparative Example 2, it is revealed that the masking layer was detached in Example 2, due to the change of the culture medium into alkaline, by injecting TMAH into the culture medium.

From the description hereinabove, many modifications and other embodiments will be apparent to persons skilled in the art. Therefore, the above description should be construed as merely illustrative exemplification, which is provided for the purpose of teaching the persons skilled in the art on the best embodiment for carrying out the present invention. Details of the structure and/or function thereof can be substantially altered without departing from the spirit of the present invention.

What is claimed is:

1. A method of the immobilization of a cell in which a cell is immobilized in a desired region on the surface of a substrate, which comprises:
the step (a) of forming a masking layer in a region except for said desired region on the surface of said substrate,
the step (b) of immobilizing said cell following the step (a) through bringing a solution containing said cells into contact with the surface of said substrate and the surface of said masking layer, and
the step (c) of adjusting the pH of said solution to give the condition which permits separation of said masking layer from said substrate without loss of the activity of said cell following the step (b).

2. The method of the immobilization of a cell according to claim 1 wherein said solution is a culture medium.

3. The method of the immobilization of a cell according to claim 1 wherein the step (a) comprises the step of forming said masking layer on the surface of said substrate, and the step of removing said masking layer in said desired region.

4. The method of the immobilization of a cell according to claim 3 wherein said masking layer is formed from a masking material having photosensitivity, and the step of removing said masking layer is the step of exposing either one of said masking layer in said desired region or in the region other than said desired region, followed by development.

5. The method of the immobilization of a cell according to claim 4 wherein the pH of said solution is elevated in the step (c), to be greater than the pH of said solution in the step (b).

6. The method of the immobilization of a cell according to claim 4 wherein said pH is adjusted to be 7.9 or greater and 8.1 or less in the step (c).

7. The method of the immobilization of a cell according to claim 1 wherein the pH of said solution is adjusted by adjusting the concentration of carbon dioxide in the ambient atmosphere of said solution in the step (c).

8. The method of the immobilization of a cell according to claim 1 wherein the pH of said solution is adjusted by adding a pH adjusting agent to said solution in the step (c).

9. The method of the immobilization of a cell according to claim 1 further comprising the step of heating said masking layer after the step (a) and before the step (b), wherein said heating is conducted at a temperature higher than the boiling point of a detrimental constituent, which is included in said masking layer, to said cell.

10. The method of the immobilization of a cell according to claim 1 wherein said cell is immobilized via an immobilization material in the step (b), and said immobilization material is a material including any one of a cell adhesive protein, a positively charged polymer, or a polymer having a strongly basic functional group.
